(19) United States
(12) Patent Application Publication
(10) Pub. No.: US 2008/0260644 A1
(43) Pub. Date: Oct. 23, 2008

(54) CHLORIDE TRANSPORT UPREGULATION FOR THE TREATMENT OF TRAUMATIC BRAIN INJURY

(76) Inventor: Akiva S. Cohen, Bala Cynwyd, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/116,588

(22) Filed: May 7, 2008

Related U.S. Application Data
(63) Continuation-in-part of application No. PCT/US2006/043618, filed on Nov. 9, 2006.
(60) Provisional application No. 60/753,521, filed on Dec. 23, 2005.
(60) Provisional application No. 60/735,138, filed on Nov. 9, 2005.

(51) Int. Cl. A61K 48/00; A61K 38/00; A61K 49/00; A61K 31/553; A61K 31/215

(52) U.S. Cl. 424/9.2; 514/2; 514/44; 424/93.2; 514/211.08; 514/506

(57) ABSTRACT

Compositions and methods are provided for the alleviation of pathology induced by traumatic brain injury.

This application is a continuation-in-part of PCT/US2006/043618, filed on Nov. 9, 2006, which claims priority under 35 U.S.C. §119(e) to U.S. Provisional Patent Application No. 60/753,521, filed on Dec. 23, 2005 and U.S. Provisional Patent Application No. 60/735,138, filed on Nov. 9, 2005. The foregoing applications are incorporated by reference herein.

Pursuant to 35 U.S.C. Section 202(c), it is acknowledged that the United States Government has certain rights in the invention described herein, which was made in part with funds from the National Institutes of Health Grant No. RO1-NS45975.

### FIELD OF THE INVENTION

The present invention relates to the alleviation of pathology induced by traumatic brain injury.

### BACKGROUND OF THE INVENTION

Every 21 seconds a person sustains a traumatic brain injury (TBI) making it a significant health issue in the United States (BIA (2004) “TBI Statistics”). TBI is a heterogeneous insult causing patients to suffer from cognitive deficits (Lyeth et al. (1990) Brain Res., 526:249-58; Cave and Squire (1991) Hippocampus, 1:329-40; Smith et al. (1991) J. Neurotrauma, 8:259-69; Gorman et al. (1993) Brain Res., 614:29-36; Zola-Morgan et al. (1993) J. Neurosci., 13:251-65; Annegers et al. (1998) N. Engl. J. Med., 338:20-4; Asikainen et al. (1999) Epilepsia, 40:584-9) and an increase in seizure frequency (Annegers, Hauser et al. 1998). Such pathological consequences are likely due to injury-induced alterations in the hippocampus (Scoville and Milner (1957) J. Neurochem., 20:11-21; Zola-Morgan et al. (1986) J. Neurosci., 6:2950-67; Annegers et al. (1998) N. Engl. J. Med., 338:20-4; Asikainen et al. (1999) Epilepsia, 40:584-9), a structure implicated in higher cognitive function (Cave and Squire (1991) Hippocampus, 1:329-40; Miller et al. (1998) Neuropsychologia, 36:1247-56). The dentate gyrus (DG) within the hippocampal formation has recently been shown to play an important role in the formation of new memories (Eldridge et al. (2005) J. Neurosci., 25:3280-6). Furthermore, robust gamma-aminobutyric acid-ergic (GABAergic) inhibition is thought to trigger filter-functions in the DG, impeding excessive or aberrant activity from propagating further into the seizure prone hippocampus (Sloviter, R. S. (1994) Ann. Neurol., 35:640-54; Heinemann et al. (1992) Epilepsy Res. Suppl., 7:273-80; Cohen et al. (2003) Eur. J. Neurosci., 17:1607-16). Following fluid percussion injury (FPI), the DG becomes more excitable (Lowenstein et al. (1992) J. Neurosci., 12:4846-4853; Witgen et al. (2005) Neuroscience, 133:1-15) likely due to a breakdown in GABAergic inhibition (Toth et al. (1997) J. Neurosci., 17:8106-8117; Witgen et al. (2005) Neuroscience, 133:1-15).

The efficacy of GABA_{A}-mediated postsynaptic inhibition depends on the maintenance of low intracellular chloride ([Cl⁻]ᵢ) concentration by the neuronal K—Cl co-transporter (KCC2) (Thompson and Gahwiler (1989) J. Neurophysiol., 61:501-33; Kaila K. (1994) Prog. Neurobiol., 42:489-537; Alvarev-Leefmans (1989) Acta Physiol. Scand. Suppl., 582:17; Rivera et al. (1999) Nature, 397:251-5). Chloride transport by KCC2 creates an inwardly directed Cl-electrochemical gradient. When the extrusion of Cl⁻ is disrupted, [Cl⁻]ᵢ increases and accumulates inside the neuron, decreasing the driving force for GABA_{A}-mediated inhibitory currents resulting in significant disinhibition (Ben-Ari et al. (1997) Trends Neurosci., 20:523-9; McCarren and Alger (1985) J. Neurophysiol., 53:557-71; Korn et al. (1987) J. Neurophysiol., 57:325-40; Alvarez-Leefmans et al. (1989) Acta Physiol. Scand. Suppl., 582:17; Huguenard et al. (1986) J. Neurophysiol., 56:1-18; Thompson et al. (1989) J. Neurophysiol., 61:501-33).

### SUMMARY OF THE INVENTION

In accordance with the instant invention, methods for reducing and/or preventing the pathology associated with traumatic injury to the brain comprising augmenting chloride transport within the brain are provided. In a particular embodiment, the method comprises augmenting chloride transport within the hippocampus or, more specifically, within the dentate gyrus.

In one embodiment, chloride transport is augmented by transiently modifying the extracellular chloride gradient by ionic substitution thereby increasing the driving force for chloride extrusion.

In yet another embodiment, chloride transport may be augmented, with or without transiently modifying the extracellular chloride gradient by ionic substitution, by increasing KCC2 function within the brain. KCC2 function may be increased by, for example, performing one or more of the following: administering an effective amount of KCC2 to the brain, administering an effective amount of a vector comprising a nucleic acid sequence coding for KCC2 to the brain, administering an agent which activates protein kinase C (PKC), and administering an effective amount of at least one brain-derived neurotropic factor antagonist.

In another embodiment, methods for screening test compounds for their ability to reduce the pathology associated with traumatic brain injury are provided.

### BRIEF DESCRIPTIONS OF THE DRAWING

FIG. 1A is an image of a representative western blot showing expression of KCC2 (142 kDa) within isolated Cornu Ammonis 1 (CA1) and dentate gyrus (DG) from ipsilateral (injured) hippocampus of both sham and fluid percussion injury (FPI) animals (n=5 per lane). β-actin (45 kDa) is provided as a control for protein loading. FIG. 1B is an image of KCC2 immunohistochemistry in the dentate gyrus of sham animals and animals 7 days following FPI. Scale bar is 10 mm. FIG. 1C is a graphical representation of real time PCR analysis of KCC2 mRNA expression in DG 7 days following FPI (N=5 per group; *=p<0.05).

FIGS. 2A, 2B and 2C are graphical representations of control (aCSF) and gamma-aminobutyric acid (GABA) currents recorded in sham (FIG. 2A), FPI (FIG. 2B), and FPI in modified (low K⁺) brain slices using a voltage ramp. Current-voltage (I-V) relationships were created by subtracting the ramp currents recorded in control (aCSF) from currents in the presence of GABA for sham and FPI brain slices. Insets are graphical representations of membrane potential in normal mature brain slices with or without bumetanide (FIG. 2A insets) and in FPI brain slices (FIG. 2B inset). The inset of FIG. 2C depicts the negative shift of EGABA when external potassium concentration is reduced from 3 to 1 mM for 3 independent neurons recorded in individual slices derived from 2 injured animals. E_{GABA} was estimated by measuring the membrane potential where the net ionic current is zero.

FIGS. 3A and 3B are graphical representations of the chloride clearance (buffering). The changes in fluorescence over time are shown for slices from naïve (FIG. 3A) and FPI (FIG. 3B) animals. Insets of FIGS. 3A and 3B demonstrate baseline fluorescence before (1), during (2), and 30 seconds after (3) GABA application.

FIGS. 4A and 4B are graphical representations of anterograde amnesia as assessed by the fear-associated freezing assay with sham animals and FPI animals. The average percent fear-associated freezing in the training context is shown in FIG. 4A. The percent freezing by minute for the 5 minute test is shown in FIG. 4B. *=p<0.05 and n=10, 11 for FPI and sham, respectively.

FIG. 5A provides images of murine brain slices which demonstrate the differences in throughput index (TI) between hippocampal slices from sham (left) and injured (right) animals. Slices were stimulated in the perforant pathway with one theta burst stimulation (TBS) (5 pulses of 50 μA at 100 Hz). Circles (kernels) represent average fluorescent signals in the DG and hilus respectively. FIG. 5B is a graph of the percent throughput of sham and injured (LFPI) mice. Mean TI in sham slices was significantly less than TI in injured slices (*p<0.05).

FIG. 6 is a graph demonstrating that phorbol 12,13-dibutyrate (PDBu) significantly (p<0.05) reduces dentate gyrus TI in slices generated from brain injured animals. Interestingly, PDBu does not affect DG filtering in slices generated from sham animals. n=21 slices from 8 mice (sham), 31 slices from 12 mice (FPI), 15 slices from 5 mice (sham with PDBU), 10 slices from 4 mice (FPI with PDBU).

### DETAILED DESCRIPTION OF THE INVENTION

Due to the importance of the hippocampus in learning and memory, seizure generation, and the damage it sustains after injury, the hippocampus is a tempting target for intervention with the possibility of developing rational, targeted therapies to minimize the incapacity and suffering caused by human TBI. In particular, within the hippocampus the DG is hypothesized to play a critical role in filtering excessive information. DG becomes hyperexcitable following FPI (Toth et al. (1997) J. Neurosci., 17:8106-8117; Witgen et al. (2005) Neuroscience, 133:1-15) and a disruption in the balance between inhibition and excitation is suggested as a responsible mechanism. Pathophysiological activity and various kinds of traumatic insults are known to have deleterious long-term effects on neuronal chloride regulation, which can lead to a suppression of fast postsynaptic GABAergic responses (Rivera et al. (2002) J. Cell. Biol., 159:747-52). However, the putative mechanism underlying this altered neuronal excitability has not been previously elucidated.

In accordance with the present invention, it has been discovered, among other things, that (1) KCC2 protein and mRNA expression are significantly reduced in DG 7 days following FPI; (2) E_{C1} of FPI brain slices are greatly depolarized compared to sham slices; and (3) reduced anterograde function and increased seizure frequency are observed post FPI.

KCC2 protein and mRNA expression have been found to be significantly reduced 7 days following FPI. The alterations present at one week post-FPI were examined because it is within the clinically relevant “therapeutic time window,” i.e., potential therapeutic intervention has the best chance of success within this time frame. It appears that in DG of injured animals, cells may not be expressing sufficient KCC2 to properly maintain the Cl⁻ gradient required for fast post synaptic inhibition. FPI-induced changes are directly translated into corresponding alterations in protein expression, indicating that this effect appears to take place at the level of transcription of KCC2, although changes at the level of regulation of KCC2 mRNA stability and/or of translation or trafficking of the KCC2 protein may also play a role. Reduced levels of KCC2 mRNA following FPI suggests that the reduction in overall protein levels may be due to a transcriptional block as opposed to a problem with translation of the mRNA to protein. Without sufficient KCC2 extrusion of Cl⁻, the intracellular Cl⁻ concentration builds to a level that diminishes the driving force necessary for efficacious inhibition.

To determine if there is an increase in the intracellular Cl⁻ concentration in DG of injured animals, the gramicidin perforated patch technique was performed. The E_{C1} values obtained using GABA in sham slices agreed with previous reported values (Owens et al. (1996) J. Neurosci., 16:6414-23; van Brederode et al. (2001) J. Physiol., 533:711-6) with a small amount of variability in studies most likely due to age and preparation (e.g., slice vs. culture). The E_{C1} values of FPI brain slices were greatly depolarized compared to those of sham slices. This appears to be due to a decrease in Cl⁻ extrusion from the cell due to the reduced levels of KCC2. An increase in intracellular Cl⁻ disrupts the Cl⁻ gradient required for the GABA_{A} receptor to function as an inhibitory signal, since it is primarily a Cl⁻ ionophore (Bormann et al. (1987) J. Physiol., 385:243-86). This decrease in GABA function provides a clear mechanism for the previously reported increased excitability in DG following injury (Lowenstein et al. (1992) J. Neurosci., 12:4846-4853; Toth et al. (1997) J. Neurosci., 17:8106-8117; Witgen et al. (2005) Neuroscience, 133:1-15).

The consequence of reduced KCC2 expression in this hippocampal subregion is twofold. First, KCC2 is chiefly responsible for maintaining low intracellular chloride essential for GABA_{A}-mediated synaptic inhibitory efficacy (Alvarez-Leefmans et al. (1989) Acta Physiol. Scand. Suppl., 582:17; Kaila K. (1994) Prog. Neurobiol., 42:489-537; Rivera et al. (1999) Nature, 397:251-5). Second, DG is important in anterograde (information encoding) cognitive function (Eldridge et al. (2005) J. Neurosci., 25:3280-6). Furthermore, robust GABA_{A}ergic inhibition is also thought to give rise to the gatekeeper function, quelling synchronous or excessive neuronal activity from propagating from the entorhinal cortex into the seizure prone hippocampus (Sloviter, R. S. (1994) Ann. Neurol., 35:640-54; Heinemann et al. (1992) Epilepsy Res. Suppl., 7:273-80; Cohen et al. (2003) Eur. J. Neurosci., 17:1607-16; Buzsaki (1983) Brain Res., 287:139-71; Lothman (1992) Epilepsy Res. Suppl., 7:301-13).

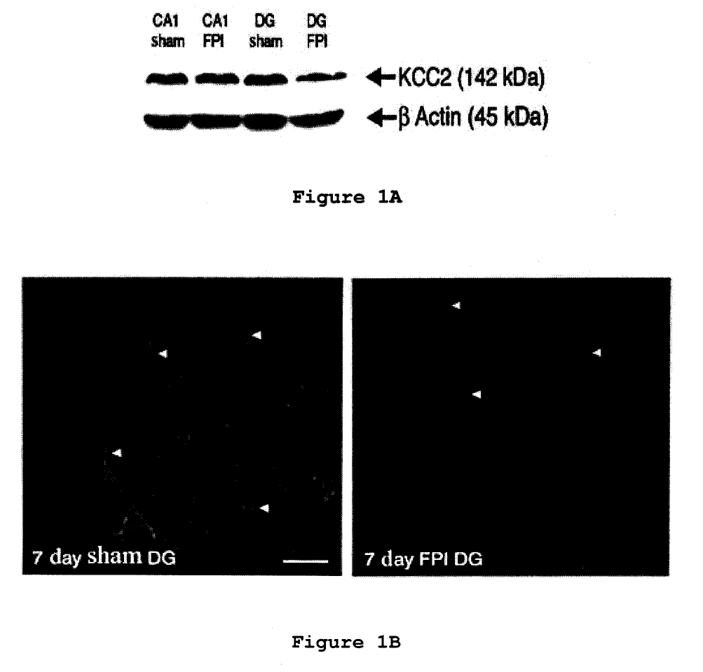

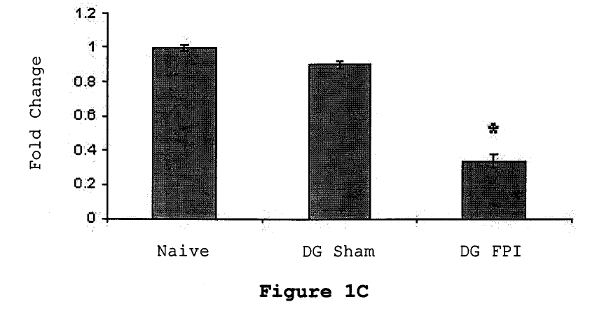

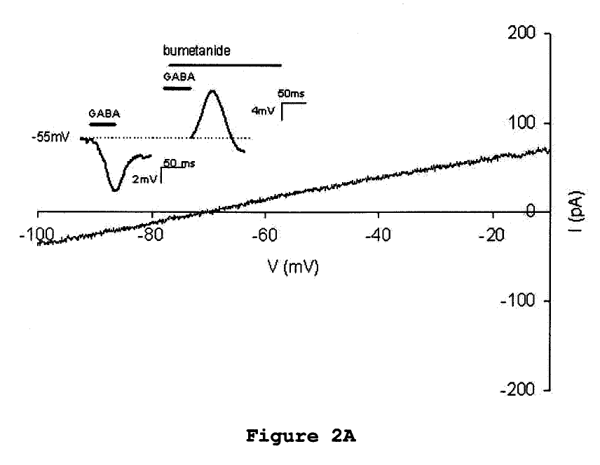

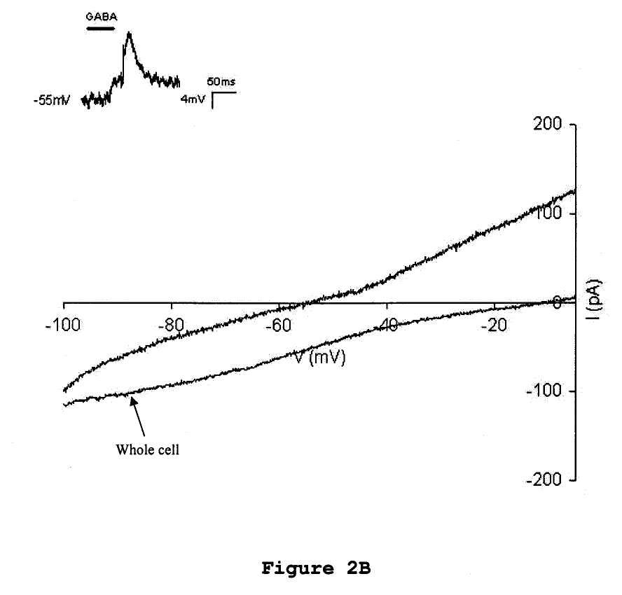

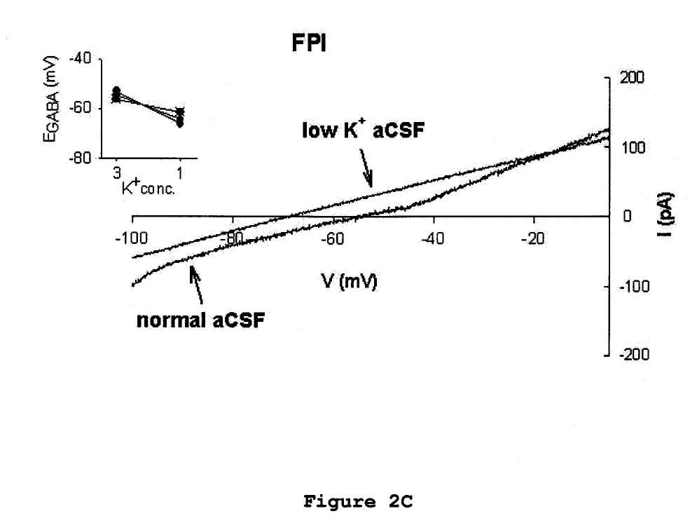

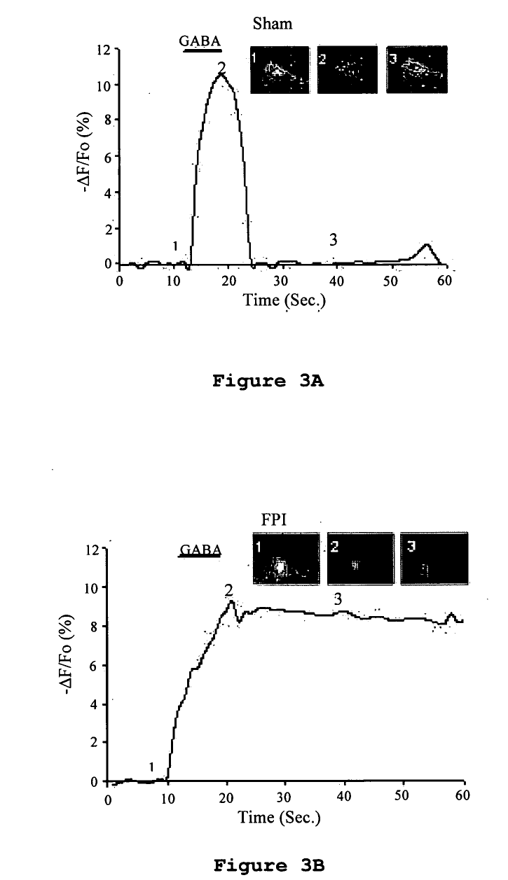

As demonstrated hereinbelow, a decrease in mRNA and protein KCC2 expression (FIG. 1) results in reduced intracellular chloride extrusion (FIG. 3) shifting E_{GABA} more positive (FIG. 2); thereby diminishing GABA_{A}-mediated inhibitory efficacy. These injury-induced alterations culminate in a cognitive impairment that has been demonstrated to rely on functional DG activity (Eldridge et al. (2005) J. Neurosci., 25:3280-6) (FIG. 3B) and is observed in human patients suffering from concussive brain injury (Zola-Morgan et al. (1986) J. Neurosci., 6:2950-67; Rempel-Clower et al. (1996) J. Neurosci., 16:5233-55).

At a minimum, the data presented herein indicate that a mild incident of FPI significantly diminishes intracellular chloride extrusion mediated by decreased KCC2 protein expression and thereby leads to a reduction in the strength of GABA_{A}ergic inhibition in DG derived from injured animals. This reduction in protein expression may take place at the transcriptional level because DG KCC2 mRNA extracted from injured animals is significantly reduced. However, alterations in translation and protein trafficking cannot be excluded.

Pathological plasticity in KCC2 has been previously demonstrated. For example, axotomy of dorsal motor neurons of the vagus in young rats (p 16-18) resulted in depolarizing GABA responses due to the decreased KCC2 expression (Nabekura et al. (2002) J. Neurosci., 22:4412-7). In vivo kindling of adult mice resulted in a significant reduction KCC2 mRNA and protein in DG (Rivera et al. (2002) J. Cell. Biol., 159:747-52). Transcortical lesioning in rat (p 21) caused an impairment of Cl⁻ extrusion resulting from decreased KCC2 expression (Jin et al. (2005) J. Neurophysiol., 93:2117-26).

In addition to decreased DG KCC2 expression, other factors in transporter function may be involved in the pathology associated with brain injuries. One potential mechanism contributing to the depolarizing E_{GABA} shift following FPI might be that brain injury reverts DG neurons to a prior developmental stage thereby upregulating developmental proteins such as NKCC1. De novo expression of NKCC1 would act to pump Cl⁻ into the cell disrupting the normal chloride gradient essential for GABAergic inhibitory function present in mature neurons. However, western blot analysis conducted in regionally dissected DG tissue isolated from sham and injured animals respectively, demonstrated no NKCC1 protein expression compared to fetal control tissue (data not shown).

The transport of K⁺ and Cl⁻ ions by KCC2 is electroneutral, thus the direction of net ionic movement is exclusively controlled by the sum of chemical potential differences of the two ions (Payne et al. (1997) Am. J. Physiol., 273:C1516-25). That is, K⁺ and Cl⁻ extrusion by KCC2 is primarily derived from cation gradients generated by the Na⁺/K⁺ ATPase. Therefore any probable injury-induced disruption in transmembrane potassium distribution or Na⁺/K⁺ ATPase (Na pump) could alter KCC2 function.

In vitro stretch injury reduced Na pump function causing a significant depolarized shift in resting membrane potentials (RMPs; Tavalin et al. (1997) J Neurophysiol., 77:632-8). This inhibition was attributed to depleted cellular energies. Interestingly, in a more intact injury model, i.e., FPI in the rat, a transient (<4 days) depolarized shift in RMPs of dentate gyrus interneurons but not dentate granule neurons was recorded in slices from injured animals (Ross et al. (2000) J Neurophysiol., 83:2916-30). Furthermore, the depolarized shift was mediated by Na pump inhibition.

In rats two days after FPI area CA1 hyperexcitability mediated by decreased glial K⁺ conductances resulting in impaired extracellular K⁺ buffering is observed (D'Ambrosio et al. (1999) J Neurosci., 19:8152-62). In contrast, FPI did not cause impaired extracellular K+ clearance at two days, one week or one month after injury (Santhakumar et al. (2003) J. Neurosci., 23:5865-76).

Therefore, even though injury-induced changes have been demonstrated in Na⁺/K⁺ ATPase activity and extracellular potassium homeostasis, these pathologies are either transient and back to pre-injury levels by 7 days post-injury or occur in brain regions other than DG. Therefore, reported alterations presented here are likely not due to Na pump dysfunction or impaired extracellular K⁺ buffering.

As stated hereinabove, DG KCC2 expression is significantly reduced one week following FPI. The significant decrease in KCC2 expression deteriorates cellular ability to maintain the transmembrane chloride gradient essential for robust GABA_{A}ergic inhibitory efficacy. Diminished inhibitory efficiency causes dysfunction of the DG contributing to observed anterograde cognitive deficits and increased seizure frequency observed following injury. Presently, few if any potential curative targets exist for TBI patients. The data indicate that KCC2 can be used for therapeutic intervention to assuage the serious ramifications of head trauma.

In accordance with the instant invention, methods for reducing and/or preventing the pathology (e.g., cognitive impairment, increased seizure rate) associated with traumatic injury to the brain comprising augmenting chloride transport within the brain are provided. In a particular embodiment, the method comprises augmenting chloride transport within the hippocampus or, more specifically, within the dentate gyrus.

Chloride transport may be augmented by increasing KCC2 function within the brain. KCC2 function, i.e., K—Cl co-transporter activity, may be increased by, for example, performing one or more of the following: administering an effective amount of KCC2 protein to the brain, administering an effective amount of a vector comprising a nucleic acid sequence coding for KCC2 to the brain, and administering an effective amount of at least one brain-derived neurotropic factor (BDNF) antagonist. BDNF activation leads to a decrease in KCC2 expression via activation of the tyrosine kinase activity of the BDNF receptor TrkB. Agents which inhibit the activation of TrkB, e.g., by blocking BDNF, prevent this decrease in KCC2 expression. Accordingly, BDNF antagonists maintain or augment KCC2 function, i.e., potassium-chloride-co-transporter activity.

In a particular embodiment, the pathology associated with traumatic brain injury is reduced or prevented by administering at least one BDNF antagonist, wherein at least one of the BDNF antagonists is K252a, TrkB-Fc, or both. In another embodiment, the pathology associated with traumatic brain injury is reduced or prevented by administering K252a, TrkB-Fc, or both.

In yet another embodiment of the instant invention, chloride transport is augmented by transiently modifying the extracellular chloride gradient by ionic substitution thereby increasing the driving force for chloride extrusion. Notably, chloride transport (efflux) is directly tethered to the potassium gradient. Accordingly, the potassium efflux can be increased by decreasing the extracellular concentration of potassium (e.g., by locally administering a biological fluid comprising a lower concentration of potassium). For example, the extracellular concentration of potassium can be reduced about 2:1, about 3:1, about 5:1, or more. As a specific example, the extracellular concentration of potassium can be lowered from about 3 mM to about 1 mM. The lowering of the extracellular concentration of potassium will serve to increase intracellular efflux from the cell.

### I. DEFINITIONS

As used herein, the term “brain-derived neurotrophic factor (BDNF) antagonist” refers to agents which inhibit activation of the BDNF receptor TrkB. Particularly, the BDNF antagonist inhibits the activation of the tyrosine kinase activity of the TrkB receptor. Exemplary BDNF agonists include TrkB inhibitors such as K252a and anti-TrkB antibodies (see, e.g., Balkowiec and Katz (2000) J. Neurosci., 20:7417-7423) and BDNF scavengers such as TrkB soluble receptors and anti-BDNF antibodies. TrkB-Fc is an exemplary TrkB soluble receptor (see, e.g., Rivera et al. (2002) J. Cell Biol., 159:747-752).

As used herein, “cognitive impairment” refers to an acquired deficit in at least one of the following: memory function, problem solving, orientation, and abstraction. The deficiency typically impinges on an individual's ability to function independently.

As used herein, the term “traumatic brain injury” includes any trauma, e.g., post-head trauma, impact trauma, and other traumas, to the head such as, for example, traumas caused by accidents and/or sports injuries, concussive injuries, penetrating head wounds, brain tumors, stroke, heart attack, meningitis, viral encephalitis, and other conditions that deprive the brain of oxygen. The traumatic brain injury may be a closed head injury which may be transient or prolonged.

The term “pathology” refers to any deviation from a healthy or normal condition, such as a disease, disorder, syndrome, or any abnormal medical condition.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, that is capable of replication largely under its own control. A replicon may be either RNA or DNA and may be single or double stranded. Generally, a “viral replicon” is a replicon which contains the complete genome of the virus. A “sub-genomic replicon” refers to a viral replicon that contains something less than the full viral genome, but is still capable of replicating itself. For example, a sub-genomic replicon may contain most of the genes encoding for the non-structural proteins of the virus, but not most of the genes encoding for the structural proteins.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g., chromatographic methods, agarose or polyacrylamide gel electrophoresis, HPLC analysis, and the like).

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

The term “pharmaceutically acceptable carrier” includes any and all solvents, dispersion media and the like which may be appropriate for the desired route of administration of the pharmaceutical composition. The use of such media for pharmaceutically active substances is known in the art. Except insofar as any conventional media or agent is incompatible with the compounds to be administered, its use in the pharmaceutical preparation is contemplated. Examples of pharmaceutically acceptable carriers include, without limitation, water, buffered saline, ethanol, polyol (for example, glycerol, propylene glycol, liquid polyethylene glycol and the like), dimethyl sulfoxide (DMSO), oils, detergents, suspending agents or suitable mixtures thereof. Suitable pharmaceutically acceptable carriers and formulations are described in Remington's Pharmaceutical Sciences, 19th Ed. (Mack Publishing Co., Easton, 1995).

The term “stabilizer” refers to a chemical agent (e.g., protein or polysaccharide) that assists to preserve or maintain the biological structure and/or biological activity of a protein. Examples of stabilizers include, without limitation, hydroxyethyl starch (hetastarch), serum albumin, gelatin, collagen, recombinant albumin, recombinant gelatin, recombinant collagen, non-oxidizing amino acid derivatives (e.g., tryptophan derivatives, such as N-acetyl-tryptophan), caprylates, polysorbates, amino acids, and divalent metal cations (e.g., Zn²⁺), and cresols.

The term “antibiotics” refers to, without limitation, β-lactams (penicillins and cephalosporins), vancomycins, bacitracins, macrolides (erythromycins), lincosamides (clindomycin), chloramphenicols, tetracyclines (e.g., immunocycline, chlortetracycline, oxytetracycline, demeclocycline, methacycline, doxycycline and minocycline), aminoglycosides (e.g., gentamicins, amikacins, and neomycins), amphotericns, cefazolins, clindamycins, mupirocins, sulfonamides and trimethoprim, rifampicins, metronidazoles, quinolones, novobiocins, polymixins and gramicidins and the like and any salts or variants thereof.

As used herein, the term “PKC activator” refers to a compound which activates/increases the phosphorylation/kinase activity of protein kinase C (PKC). PKC activators include, without limitation, phorbol compounds (esters) (e.g., 12-O-tetradecanoylphorbol-13-acetate (TPA), phorbol-12,13-dibutyrate (PDBu), phorbol 12-myristate 13-acetate (PMA), phorbol 12,13-didecanoate (PDD), croton extracts (e.g., leaves seeds, oils), see, e.g., Castagna et al. (1982) J. Biol. Chem., 257:7847; Nishizuka (1984) Nature, 308:693; Nishizuka (1984) Science, 225:1365; Niedel et al. (1983) Proc. Natl. Acad. Sci., 80:36, Nishizuka (1986) Science, 233:305, Blumberg (1980) Crit. Rev. Toxicol., 8:153), benzolactams (e.g., (2S,5S)-8-(1′-decynyl)benzolactam), pyrrolidones analogs (see e.g., U.S. Patent Application Publication No. 2004/0019018), bryostatins (e.g., byrostatin 1; see U.S. Patent Application Publication No. 2007/0270485), teleocidins, gnidimacrin, 6-(N-decylamino)-4-hydroxymethylindole (DHI), mezerein, N-Heptyl-5-chloronaphthalene-1-sulfonamide, farnesyl thiotriazole, (−)-indolactam V, (−)-7-octylindolactam V, ingenol 3,20-dibenzoate, lyngbyatoxins, iripallidal, aplysiatoxins, diacylglycerols (e.g., 1,2-dioctanoyl-sn-glycerol (DOG), 1-oleoyl-2-acetyl-sn-glycerol (OAG); see also Niedel et al. (1983) Proc. Natl. Acad. Sci., 80:36; Mori et al. (1982) J. Biochem., 91:427; Kaibuchi et al. (1983) J. Biol. Chem., 258:6701; Kerr et al. (1987) Biochem. Biophys. Res. Commun., 148:776; Go et al. (1987) Biochem. Biophys. Res. Commun., 144:598; Shinomura et al. (1991) Proc. Natl. Acad. Sci., 88:5149), and analogs and derivatives thereof.

### II. ADMINISTRATION

The agents of the instant invention used to augment chloride transport within the brain will generally be administered to a patient as a pharmaceutical preparation. The term “patient” as used herein refers to human or animal subjects.

The pharmaceutical preparation comprising at least one of the agents of the instant invention may be conveniently formulated for administration with a pharmaceutically acceptable carrier. Solubility limits of the agents within the particular pharmaceutically acceptable carrier may be easily determined by one skilled in the art.

Selection of a suitable pharmaceutical preparation depends upon the method of administration chosen. In this instance, preferred pharmaceutical preparations comprise at least one agent of the instant invention dispersed in a pharmaceutically acceptable carrier that is compatible with the brain. The methods of the instant invention comprise the administration of compositions for augmenting chloride transport within the brain directly into the brain of the injured subject soon after injury. Typically, the compositions will be administered directly to the brain using techniques known to those skilled in the art. The compositions may be administered specifically to the hippocampus, more specifically to the dentate gyrus.

Methods for determining the most effective means and dosage of administration are well known to those of skill in the art and will vary with the vector used for therapy (e.g., gene therapy), the nucleic acid used for therapy, the polypeptide used for therapy, the purpose of the therapy, the target cell being treated, and the subject being treated.

The dose and dosage regimen of a pharmaceutical preparation may be determined by a physician considering the patient's age, sex, weight, general medical condition, and the specific condition and severity thereof for which the preparation is being administered. The physician may also consider the route of administration of the agent, the pharmaceutical carrier with which the agent may be combined, and the agent's biological activity.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Appropriate concentrations for alleviation of a particular pathological condition may be determined by dosage concentration curve calculations, as known in the art

In accordance with the present invention, the appropriate dosage unit for the administration of agents of the instant invention may be determined by evaluating the toxicity of the agents in animal models. Various concentrations of pharmaceutical preparations may be administered to mice having sustained a traumatic brain injury and the minimal and maximal dosages may be determined based on the results of significant reduction of pathology as a result of the treatment. Appropriate dosage unit may also be determined by assessing the efficacy of the treatments in combination with other drugs.

Administration may be effected continuously or intermittently throughout the course of treatment. The pharmaceutical preparation of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient. Administration can be achieved by any known method or mechanism, such as, by use of a syringe, infusion pump (e.g., implantable pumps), cannulas, and catheters.

In a particular embodiment, the pharmaceutical preparations of the instant invention are administered immediately or soon after the traumatic brain injury event. Preferably, the pharmaceutical preparation is administered at least within about the first 7 days after injury, within about the first day after injury, within about the first hour after injury.

As used herein, a “therapeutically effective amount” of an agent or composition of the present invention is an amount sufficient to modulate pathology associated traumatic brain injury in a patient.

The compositions of the instant invention can be formulated in pharmaceutically acceptable compositions suitable for delivery to the brain. In general, the formulations may contain other components in amounts that do not detract from the preparation of effective safe formulations. The compositions of the instant invention may further comprise at least one preservative, stabilizer, carriers, excipients, and/or antibiotic. Useful carriers for agents of the present invention may include, without limitation, any artificial or natural lipid-containing target molecule, cellular membranes, liposomes, and micelles. In a particular embodiment, compositions of the instant invention can be formulated as a liquid solution or suspension suitable for use with syringes, infusion pumps (e.g., implantable pumps), cannulas, and catheters.

The pharmaceutical preparations of the instant invention may be administered in vivo or ex vivo. In a particular embodiment, the vectors encoding KCC2 may be administered in solution directly to the brain or may be administered to cells outside of the body which are then transplanted into the brain. The cells treated ex vivo may be obtained from the patient or may be neuron or neuron-like cell lines compatible with transplantation into the brain (e.g., the PC12 cell line). The cells may be prepared for implantation by suspending the cells in a physiologically compatible carrier, such as cell culture medium or phosphate buffered saline. The volume of cell suspension to be implanted will vary depending on the site of implantation, and cell density in solution. In a particular embodiment of the invention, a cell suspension of 10,000 to 25,000 cells may be administered in each injection. Several injections may be used in each host. Persons of skill in the art will be able to determine proper cell dosages for this purpose.

In another embodiment of the invention, tissue fragments or “patches” of cells may be implanted into the brains of patients. Exemplary tissue fragments may be 100 to 200 μm in diameter. The cells of the invention may also be encapsulated by membranes prior to implantation. The encapsulation provides a barrier to the host's immune system and inhibits graft rejection and inflammation. Several methods of cell encapsulation are known in the art such as those described in U.S. Pat. Nos. 4,353,888, 4,744,933, 4,749,620, 4,814,274, 5,084,350 or 5,089,272, each of which is incorporated by reference herein. The cells of the invention will be administered to the hosts by surgical implantation or grafting into the brain. Suitable methods for transplantation of cells into the brains of patients having neurological disorders are provided in U.S. Pat. Nos. 5,869,463 and 5,690,927, the entire disclosures of each being incorporated by reference herein. In an exemplary method of the invention, the cells may be grafted within the hippocampus or the dentate gyrus. An exemplary method of cell transplantation has been described previously by Freed et al. (N. Engl. J. Med. (2001) 344:710-719). The transplant procedure may be performed while the host is awake, with local anesthesia administered, in order to permit surgeons to assess the host's ability to speak and to move during the transplant procedure. After the surgery, the host may optionally be administered immunosuppressive drugs to prevent rejection of the implanted cells.

### III. ANIMAL MODEL

The instant invention also provides a murine model wherein injury-induced neuronal loss and altered hippocampal regional excitability can be assessed with injury-induced cognitive deficits in the same animal set. The instant model allows for the determination of cellular, circuit and synaptic alterations in regions of the brain (e.g., hippocampus, DG, hilus, CA3, CA1, among others) that may contribute to traumatic brain injury (TBI)-induced cognitive impairment. Exemplary observations (e.g., pathologies) related to the brain injury that can be made with the murine model include, without limitation, cognitive deficiency (e.g., amnesia, fear response, fear-associated memory); neuronal loss within regions of the brain; modulation (increase or decrease) of net synaptic efficacy within regions, regional shifts in excitability and, optionally, its reversibility; changes in action potential-dependent and independent inhibitory neurotransmission in cells of particular regions; changes in ion (e.g. chloride) transport and/or concentration; and modulation in KCC2 function and/or expression. In a particular embodiment, the effect of the test compound is observed at least with the injury-induced cognitive effects. In yet another particular embodiment, the effect of the test compound is observed at least with the modulation (e.g., decrease) of KCC2 function, phosphorylation, and/or expression in the brain, particularly in the hippocampus and/or dentate gyrus.

The mice of the instant model are administered a traumatic brain injury. The traumatic brain injury may be administered by fluid percussion injury, particularly by the methods set forth below. The severity of the injury can be modulated from mild to moderate to severe. Further, while mice are preferred as models, other animals, particularly mammals other than humans, can be used.

The models of the instant invention can be used to screen compounds for their ability to modulate the effects observed in the mice after the brain injury (e.g., the pathology associated with brain injury). Any mode of administration may be used for the test compound including, for example, systemic and direct local administration. The test compound may be administered prior to the brain injury to determine the ability of the test compound to lessen the negative effects (e.g., pathology) associated with the brain injury (e.g., to test the preventative nature of the compound). The test compound may also be administered after the brain injury to the subject to determine the ability of the test compound to reduce the negative effects associated with the brain injury or prevent the onset of the negative effects.

The following examples describe illustrative methods of practicing the instant invention and are not intended to limit the scope of the invention in any way.

### EXAMPLE 1

### Materials and Methods

### Generation of Traumatic Brain Injury (TBI) Animals

Lateral fluid percussion injury (FPI) was performed as follows. On day 1, each adult male C57BL/6J mouse (5-7 weeks, 20-25 g; Jackson Laboratory, Bar Harbor, Me.) was anesthetized using sodium pentobarbital (65 mg/kg, i.p.). The animal was placed in a mouse stereotaxic frame (Stoelting, Wood Dale, Ill.). The scalp was reflected with a single incision and the fascia scraped from the skull. All of the following procedures were conducted under 0.7-3.5× magnification. An ultrathin Teflon disc, with the outer diameter equal to the inner diameter of a trephine, was glued with Vetbond (3M, St. Paul, Minn.) onto the skull between Lambda and Bregma, and between the sagittal suture and the lateral ridge over the right hemisphere. A miniature screw was placed into the skull directly above the right olfactory bulb. Using a trephine (3 mm outer diameter), the craniectomy was performed, keeping the dura intact. A rigid Luer-loc needle hub (3 mm inside diameter; Becton Dickinson, Franklin Lakes, N.J.) was secured to the skull over the opening with cyanoacrylate adhesive and dental acrylic. The skull sutures were sealed with the cyanoacrylate during this process to ensure that the fluid bolus from the injury remained within cranial cavity. The hub was capped until day 2. The animal was sutured, placed on a heating pad, and returned to the home cage once ambulatory.

On day 2, each animal was placed under isoflurane anesthesia (2% oxygen in 500 ml/minute) via nose cone and respiration was visually monitored. Once the animal reached a surgical plane of anesthesia (one respiration per 2 seconds), the nose cone was removed, the cap over the hub was removed, and dural integrity was visually confirmed. The hub was filled with isotonic sterile saline and a 32 cm piece of high-pressure tubing from the FPI device (Department of Biomedical Engineering, Virginia Commonwealth University, Richmond, Va.) was attached to the Luer-loc fitting of the hub. The animal was placed onto a heating pad on its left side and, once a normal breathing pattern resumed, before sensitivity to stimulation, the injury was induced by a 20 ms pulse of saline onto the dura. The pressure transduced onto the dura was monitored with an oscilloscope, with injury severity ranging between 2.0 and 2.1 atm. Immediately after injury, the hub was removed from the skull and the animal was placed in a supine position. Previous studies in rats have shown that the duration of unresponsiveness after experimental brain injury correlates with the degree of histopathology (Morehead et al. (1994) J. Neurotrauma, 11:657-667). In the study, the time elapsed until the animal spontaneously righted was recorded as an acute neurological assessment, and defined as the righting reflex time. Animals in the sham group had mean righting times less than 20 seconds (n=22). Injured animals were included in the study only if righting times were greater than 250 seconds (n=22). A mild to moderate injury was selected by excluding animals which died after FPI, animals that had insufficient righting times after injury, and animals that had an excessive righting time after sham surgery. The animal was then anesthetized under isoflurane to suture the scalp. Sham animals received all of the above, with the exception of the fluid pulse. Animals were returned to a heating pad until ambulatory and then returned to the home cage.

All injured mice received a fluid pulse of 2.1 atm that under our conditions constitutes a mild to moderate brain injury. Sham controls underwent all steps of FPI procedure, with the exception of the fluid pulse. All experimental procedures and protocols for animal studies were approved by the Children's Hospital of Philadelphia, Institution for Animal Care and Use Committees in accordance with international guidelines on the ethical use of animals. Experiments were designed to minimize the number of animals required and those used were cared for, handled and medicated as appropriate to minimize their suffering (National Research Council, National Academy Press, Washington, D.C., 1996).

### Sub Regional Dissection of Hippocampus

Seven days post-FPI, the ipsilateral hippocampus from sham and injured animals was harvested and sectioned using a Brinkmann tissue chopper. Sections are oriented on the dissecting surface such that the pyramidal cell layer, hippocampal fissure, and supra- and infra-granular blades of dentate gyrus (DG) are clearly visible. CA1 and DG are then micro dissected and the tissue is subsequently placed into lysis buffer containing protease inhibitor cocktail tablets (Roche Indianapolis, Ind.). To obtain sufficient tissue necessary for these experiments, regionally dissected DG and CA1 were pooled from five sham and FPI animals, respectively.

### Western Blotting

Membrane proteins (50 μg) were separated by SDS-polyacrylamide gel electrophoresis, electrophoretically transferred to polyvinylidene difluoride (PVDF) membrane (Bio Rad, Hercules, Calif.) and immunoblotted with a rabbit polyclonal antibody against KCC2 (1:1000) at 4° C. overnight (Upstate, Lake Placid, N.Y.). Membranes are then washed and incubated with a secondary antibody (1:3000) followed by chemiluminescence detection (Pierce, Rockford, Ill.). Membranes are stripped and re-probed using a rabbit polyclonal beta-actin antibody (Santa Cruz Biotechnology, Santa Cruz, Calif.) to ensure proper protein loading. Densitometry was conducted using UN-SCAN-IT gel-scanning software (Silk Scientific, Orem, Utah) to quantify and statistically compare protein expression between injured and sham populations.

### Immunostaining

Animals were intra cardially perfused with 4% PFA (n=4 animals per group). Fifty μm sections were incubated in KCC2 primary antibody at 4° C. overnight before being incubated for 1 hour at room temperature in a 1:1000 dilution of FITC conjugated goat anti rabbit secondary (Jackson Immunoresearch, West Grove, Pa.). Immuno labeling was analyzed using a Leitz Diaplan microscope equipped for epifluorescent illumination. Images were collected using a Leica DC 300 FX digital camera.

### Real Time PCR

Seven days after injury, hippocampi were harvested from sham and FPI animals (n=5 for both groups) and quickly placed into ice cold RNAlater (Qiagen, Valencia, Calif.) to stabilize and protect the cellular RNA. Micro dissection was done in an identical fashion as described above, except tissue was dissected in ice cold RNAlater. RNA was isolated using an RNeasy Lipid tissue isolation kit (Qiagen). Approximately 50 ng of RNA template, per reaction, was used to run a one step RT-PCR using the ABI PRISM 7500 sequence detection system (Applied Biosystems, Foster city, CA; GenBank Accession No. NM_{—}020333). All samples were run in triplicate including standard curves, NTC (no template control), naïve, and normalized to the beta actin endogenous reference gene. All samples were normalized and calibrated to naive.

### Electrophysiology

Brain slices were prepared as previously reported (Witgen et al. (2005) Neuroscience, 133:1-15). Perforated patch recordings were conducted at room temperature from visually identified DG neurons using infrared differential interference contrast video microscopy (Stuart et al. (1993) Pflugers Arch., 423:511-8). Cells were voltage clamped at −60 mV, and signals were recorded and amplified with Axopatch 200 (Axon instruments, Foster city, CA), filtered at 2 kHz, digitized, and stored on a PC microcomputer for off line analysis. Electrodes were pulled to a resistance between 6 and 8 MΩ when filled with an internal solution composed of in (mM): 100 KCl, 10 Hepes, pH 7.4 (KOH), on a two stage puller (Sutter instruments, Novato, Calif.). Gramicidin (Sigma, St. Louis) was dissolved in dimethyllsulfuxide (DMSO, Sigma) (1-2 mg/ml) then diluted in the pipette filling solution (1:1000). Progress of membrane patch perforation was evaluated by following the decrease in access resistance (Ra) and recordings were begun once Ra had stabilized (17-45 mΩ). Current-voltage (IV) relationships were generated using a voltage ramp (−110 to +40 ata rate of 107 mV/ms) in the absence and presence of agonist (GABA, 100 μM, applied 125 ms, 40-60 PSI using a Picospritzer II, General Valve Corporation, Fairfield, N.J.). For chloride fluorescence experiments, the internal solution was composed of (in mM) Kgluconate, 145; MgCl₂, 2; BAPTA, 0.1; KCl, 2.5; NaCl, 2.5; HEPES, 10; GTP-Tris, 0.5; Mg-ATP, 2; pH 7.4 (KOH; V_{hold}=0 mV). For low K⁺ experiments, KCl concentration was reduced from 3 to 1 mM while the concentration of NaCl was increased to maintain tonicity.

### Chloride Fluorescence

The fluorescent chloride indicator 6-methoxy-N-ethylquinolinium iodide (MEQ, 100 μM) was included in the recording pipette in order to load DG neurons in slices derived from injured and sham animals. The Cl⁻ indicator MEQ was excited by single wavelength illumination from an argon lamp band pass (340-480 nm) filtered and emitted fluorescence images (filtered at 435-485 nm) were captured with a CCD camera (Hamamatsu, Orca, Japan) and stored on a PC microcomputer. Images were subsequently analyzed offline with image pro software (Media Cybernetics, Silver Spring, Md.). For the analysis of fluorescence changes in each region of interest (ROI, i.e., patched neuron), the index −ΔF/F, was used to estimate relative change of intracellular Cl⁻ concentration. Background fluorescence (F) was the averaged fluorescent intensity obtained for two seconds before the stimulation, and FΔF was the increase from the value of F to fluorescent intensity excited at a given time. Absolute Cl⁻ concentration was not determined in the present study.

### Contextual Fear Response

Classical conditioning experiments are performed using the fear conditioning as previously described (Witgen et al. (2005) Neuroscience, 133:1-15). To assay anterograde learning, mice were brain-injured, trained in the conditioning chamber at 7 days post-injury, and then tested 24 hours later for recall of the association. Recall is assayed by scoring freezing behavior at 5 second intervals over the 5 minute duration of the assay. Percent freezing is defined as number of “frozen” observations divided by total observations.

### Reagents and Statistical Tests

Reagents were purchased from the following vendors: all salts, Gramicidin, tetrodotoxin (TTX), and GABA from Sigma (St. Louis, Mo.); CGP 55845 from Tocris (Ellisville, Mo.). All drugs were made as stock solutions and then diluted to their final concentration in the bathing medium. Two tailed unpaired Student's t tests were performed to determine statistical significance at the p<0.05 confidence level when comparing different treatment groups. All data are presented as group means ±SEM (unless otherwise noted).

### Results

To begin to elucidate a possible mechanism underlying injury-induced increased dentate gyrus net synaptic efficacy, KCC2 protein expression was examined in regionally dissected DG and area CA1 (n=5 per lane) using western blot analysis (FIG. 1A). Expression of KCC2 was robust in both regions dissected from sham animals FIG. 1A, lanes 1 & 3). Interestingly, KCC2 protein expression was unaltered in area CA1 but significantly reduced in DG dissected from ipsilateral (injured) hippocampi of animals 7 days following FPI (FIG. 1A, lanes 2 & 4). Dentate gyrus KCC2 protein expression derived from injured animals was approximately 44% (by densitometery) of that observed in DG derived from sham animals (FIG. 1A, p<0.05, westerns of pooled tissue run in triplicate). FPI induced a reduction in KCC2 protein expression in DG but not in area CA1.

As a corollary to western blot analysis, it was hypothesized that fluid percussion injury would result in a reduction in perisomatic KCC2 immunoreactive staining. KCC2 immunoreactivity was detected as densely packed perisomatic puncta surrounding neuronal somata in the dentate granule cell layer (FIG. 1B, sham panel). As predicted, less immunoreactivity and intensity surrounding dentate granule somata is clearly evident in hippocampal slices derived from injured animals compared to that observed in slices generated from sham animals (FIG. 1B, FPI panel).

To determine whether the reduction in KCC2 protein expression in DG from injured animals was due to a reduction in the amount of mRNA, real-time RT PCR was performed. Samples were normalized, calibrated to DG mRNA from naïve animals and expressed as fold change, compared to expression in naïve tissue. When compared to mRNA from sham animals (n=5), there was a 62.2% reduced expression of KCC2 DG mRNA from injured animals (FIG. 1C, n=5, p>0.05). Sham surgery was without effect on KCC2 mRNA expression as KCC2 mRNA levels from sham and naïve animals were not significantly different (FIG. 1C, p>0.05).

In order to investigate the functional consequences of reduced KCC2 expression in DG from injured animals, gramacidin perforated patch recordings were performed in dentate granule cells in brain slices derived from sham and FPI animals. Current-voltage (1-V) relationships were created using a voltage ramp that stepped the membrane voltage from a holding potential of −60 mV to −110 mV for 200 ms and then ramped the voltage from −110 to +40 mV at a rate of 107 mV/msec. I-V relationships were plotted by subtracting ramp currents recorded in control solution (aCSF) from currents recorded in the presence of ionophoresed agonist (GABA 100 μm) in brain slices from both sham (FIG. 2A) and FPI (FIG. 2B) animals. E_{GABA} values of naïve and sham animals were not significantly different and thus pooled. E_{GABA} values of FPI animals were significantly shifted toward depolarizing membrane potential. Mean E_{GABA} values were −72±4.3 (n=7) in sham slices and −51±4.0 mV (n=16) in FPI slices. To ensure that the perforated patch configuration remained intact during the experiment, in several dentate granule cells (recorded in slices from both sham and injured animals) the patch was subsequently ruptured to enter into the whole cell configuration. For both sham and FPI slices the E_{GABA} was shifted more positive −2.3±1.6 mV (n=18), close to the predicted value (−5 mV) due to the dialysis of the high chloride content present in the electrode. E_{GABA} recorded in area CA1 pyramidal neurons in slices from injured animals was −75±5.9 (n=4, not significantly different from CA1 values in slices from sham animals, p>0.05) and similar to previous reported control values, substantiating the western blot data (FIG. 1) and demonstrating specificity for DG. In all DG neurons derived from naïve or sham animals, iontophoresed GABA (100 μM) always resulted in a hyperpolarizing response (−14±0.7 mV, FIG. 2A inset). However, in DG neurons recorded in slices derived from injured animals ionotophoresed GABA resulted in an expected depolarizing response due to the hypothesized increase in intracellular Cl⁻ concentration and recorded shift in EGABA (FIG. 2B inset). In order to validate that a reduction in KCC2 underlies the observed shift in EGABA and switched GABA from an inhibitory to excitatory transmitter, bumetanide, which has been demonstrated to inhibit KCC2 (Payne et al. (1997) Am. J. Physiol., 273:C1516-25) was bath applied to slices from sham animals. In the presence of bumetanide (10 μM) focally applied GABA now resulted in a significantly smaller hyperdepolarizing response (39% compare to GABA alone, −5±3.1 mV, n=15 and several depolarizing responses, n=5, FIG. 2A inset) similar to that observed in DG neurons recorded in slices from injured animals (FIG. 2A inset).

Since the fidelity of KCC2 transport relies on the electrochemical gradient for both potassium and chloride, it was examined whether altering the driving force of potassium by external ionic substitution would lead to a restorative (negative) shift in E_{GABA} recorded in DG from injured animals. After recording E_{GABA} in DG neurons in slices from injured animals in normal aCSF ([K⁺]ₒ=3 mM), the superfusing external was switched to low [K⁺]ₒ (1 mM) aCSF. The subsequently recorded EGABA was more negative compared to the value recorded in normal aCSF (FIG. 2C and inset, mean shift of −8±1.5 mV, n=3). Furthermore, E_{GABA} recorded in low K⁺ external was significantly more negative compared to values obtained in normal external (−66.0±2.26, n=6, −51±4.0 mV, n=16 p<0.05). These data suggest that the efficacy of GABA-mediated inhibition can be partially restored by reducing [K⁺]ₒ.

To further examine the functional consequences of reduced DG KCC2 expression in DG neurons the fluorescent chloride indicator (MEQ) was loaded into cells via patch pipettes to examine Cl⁻ buffering. It was imperative to establish the intracellular Cl⁻ concentration at the same initial level, since DG neurons from injured animals would be expected to have higher resting intracellular Cl⁻ levels (due to reduced KCC2 expression). Therefore a low chloride intracellular solution was used and GABA (30 μM) was bath applied for 30 seconds to equivalently chloride load the neurons. Quantification of chloride clearance (buffering) was accomplished by measuring the fluorescence decay. The decay time was significantly longer in DG neurons recorded in slices from injured animals compared to those recorded in DG neurons from sham slices (FIG. 3A; 6±1.5; >60 sec, n=8, 16, for sham and FPI respectively, p<0.05). Furthermore, the rate of rise of the Cl⁻ transient was significantly slower in neurons recorded in slices from injured animals (FIG. 3B; 3±5.8, 2±1.9 sec, n=8, 16 for sham and FPI respectively, p<0.05).

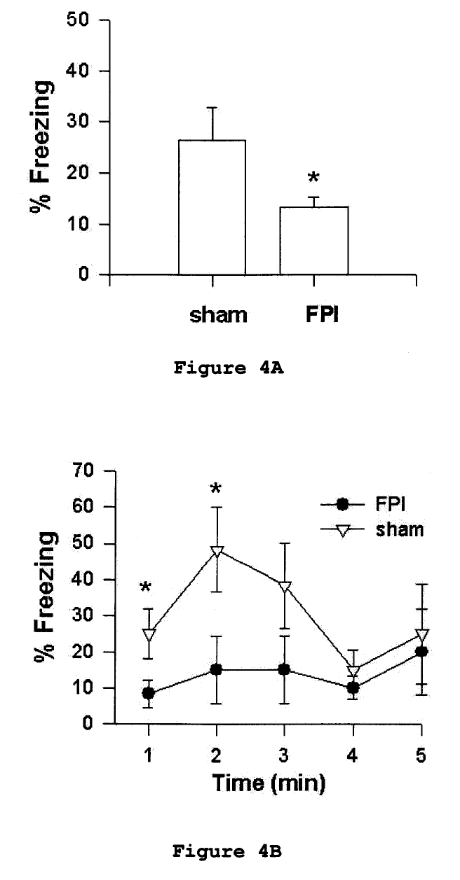

Formation or encoding of new information is thought to be dependent on proper dentate gyrus information processing (Eldridge et al. (2005) J. Neurosci., 25:3280-6). Therefore, it was hypothesized that injured animals would demonstrate anterograde cognitive deficits due to dysfunctional DG information processing induced by altered inhibitory efficacy. To test this hypothesis, injured and sham animals were trained and tested using conditioned fear paradigm to assay for injury-induced anterograde cognitive impairment one-week post-FPI. As predicted, injured animals demonstrated anterograde cognitive impairment by significantly less fear-associated freezing compared to that exhibited by sham animals (FIGS. 4A and 4B; n=10, 11 for FPI and sham respectively, p<0.05). As a control, both injured and sham treated mice demonstrated similar freezing behavior when challenged with a novel context, suggesting that the FPI selectivity alters the animals' cognitive ability when challenged with a DG-dependent task.

### EXAMPLE 2

Normal hippocampal function is directly determined by a balance between neuronal excitation and inhibition. Within the hippocampus there are three interconnected subregions referred to collectively as the ‘Trisynaptic Circuit.’ This circuit consists of the dentate gyrus (DG), area CA3, and area CA1, each having distinct physiological roles. The DG “filters” out aberrant or excessive input to the hippocampus and limits this activity from propagating further along the hippocampal circuit. Area CA3 then “amplifies” the activity that the DG has allowed to enter, while area CA1 “transduces” processed hippocampal information outflow to the cerebral cortex.

It is believed that the DG acts as a filter because it is extraordinarily resistant to the generation of synchronized bursting characteristic of epileptic seizures. This dampening of synaptic input may be critical in protecting the exquisitely fragile neurons of the hippocampus proper from excessive activation. The filtering behavior of the DG is due to two main mechanisms: the intrinsic properties of dentate granule cells (DGCs), which resist hyperexcitability, and network properties within the DG, which restrict synchronized bursting of DGCs. The filter's efficacy is further enhanced by robust surround (feedforward and feedback) inhibition. Making use of fluorescent voltage sensitive dyes as a measure of hippocampal neuronal activity, a straightforward relationship has been devised in order to quantify the efficacy of dentate gyrus filtering capacity. “Throughput Index” (TI) is defined as the amount of fluorescence in the hilus divided by the fluorescence in the upper (suprapyramidal) blade of the dentate gyrus induced by afferent (perforant path) stimulation.

TI=(Hilus_{fluorescence}/Supra DG_{flourescence})*100

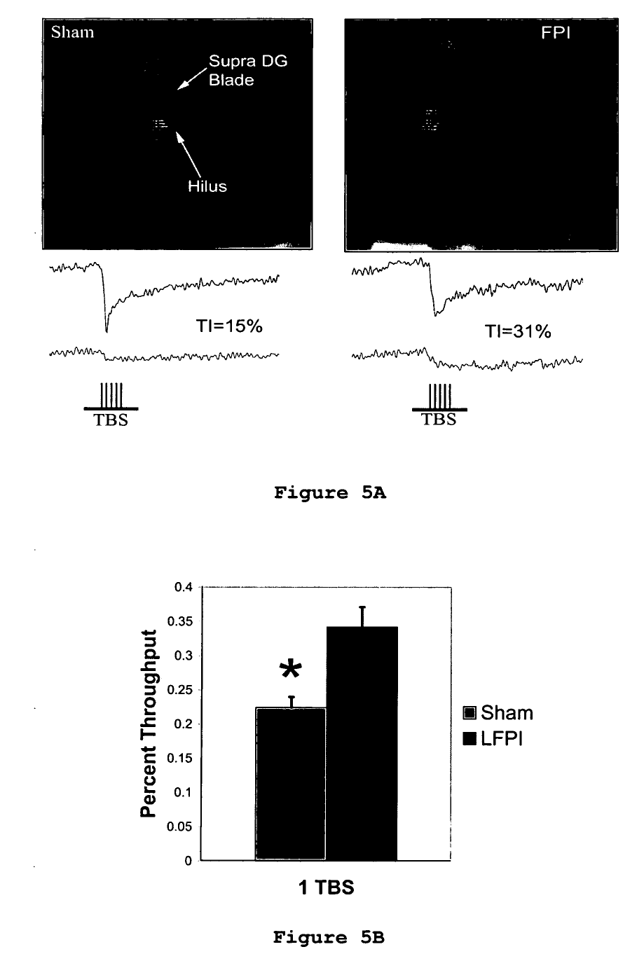

As a means of simplification: as TI increases, DG filtering decreases thus contributing to post-traumatic seizures. TI calculated in slices from sham animals was 22.4±0.1% (FIG. 5, n=31 slices, 11 animals) and significantly less than 34.2±0.2% calculated in slices from injured animals 7 days post lateral fluid percussion injury (LFPI) (p<0.05, FIG. 5, n=26 slices, 12 animals) demonstrating that DG filtering is diminished by brain injury.

Inhibition can be thought of as a two component process. First and foremost, there is driving the membrane potential further away from action potential firing threshold (hyperpolarization). The second component is shunting depolarizing current. The first component is crucially dependent on the maintenance of a low intracellular chloride concentration ([Cl⁻]ᵢ). As stated herein, mature dentate gyrus neurons regulate [Cl⁻]ᵢ via a neuronal K—Cl co-transporter (KCC2) driven by the transmembrane potassium gradient for ionic transport. When chloride extrusion is disrupted due to decreased KCC2 expression, [Cl⁻]ᵢ increases inside the neuron, decreasing the driving force for GABAA-mediated inhibitory potentials resulting in significant disinhibition (increased excitability). Significantly, genetic manipulations resulting in decreased levels of KCC2 expression increase susceptibility to seizures. Further, a dentate gyrus specific decrease in KCC2 expression in brain injured mice 7 days post LFPI has been demonstrated. This decrease in KCC2 expression results in abridged intracellular chloride extrusion reducing transmembrane chloride gradient. This diminishes dentate gyrus inhibitory efficacy and, thus, contributes to compromised DG filtering ability. Therefore, exogenously regulating the level of KCC2 phosphorylation/dephosphorylation may prolong the lifetime of active KCC2 in the lipid bilayer thereby reducing throughput index.

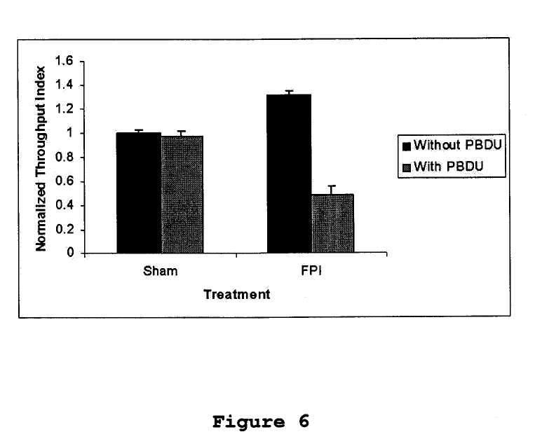

Phosphorylation/dephosphorylation directly impacts KCC2 lifetime in the plasma membrane. Dephosphorylation of KCC2 leads to internalization of the protein and phosphorylation stabilizes membrane surface expression of KCC2. Recent evidence supports this approach demonstrating that direct protein kinase C (PKC) dependent phosphorylation of KCC2 serine⁹⁴⁰ increases KCC2 membrane lifetime by decreasing the rate of KCC2 internalization (Lee et al. (2007) J. Biol. Chem., 282:29777-29784). Phorbol 12,13-dibutyrate (PDBu), which specifically activates PKC and indirectly phosphorylates KCC2, significantly reduces throughput index (TI) in slices generated from injured animals (see FIG. 6) substantiating the position that reagents designed to phosphorylate KCC2 may be used to raise reduced seizure threshold in brain injured patients.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for reducing in a patient the pathology associated with traumatic injury to the brain comprising augmenting chloride transport within the brain.

2. The method of claim 1, wherein said augmenting chloride transport within the brain comprises increasing KCC2 function within the brain.

3. The method of claim 2, wherein said increasing KCC2 function comprises administering an effective amount of KCC2 to the brain.

4. The method of claim 3, wherein said effective amount of KCC2 is administered to the hippocampus.

5. The method of claim 4, wherein said effective amount of KCC2 is administered to the dentate gyrus.

6. The method of claim 3, wherein said effective amount of KCC2 is administered by stereotactic injection.

7. The method of claim 2, wherein said increasing KCC2 function comprises administering an effective amount of vector comprising a nucleic acid sequence coding for KCC2 to the brain.

8. The method of claim 7, wherein said vector is a plasmid.

9. The method of claim 7, wherein said vector is a viral vector.

10. The method of claim 7, wherein said vector is expressed in a cell suitable for transplantation and said cells are transplanted into said patient.

11. The method of claim 2, wherein said increasing KCC2 function comprises administering an effective amount of at least one brain-derived neurotropic factor antagonist.

12. The method of claim 11, wherein said at least one brain-derived neurotropic factor antagonist comprises K252a.

13. The method of claim 11, wherein said at least one brain-derived neurotropic factor antagonist comprises TrkB-Fc.

14. The method of claim 11, wherein said at least one brain-derived neurotropic factor antagonist comprises K252a and TrkB-Fc.

15. The method of claim 1, wherein said pathology is selected from the group consisting of cognitive impairment and reduction of seizure threshold.

16. The method of claim 1, wherein said augmenting chloride transport within the brain comprises transiently modifying the extracellular chloride gradient by ionic substitution thereby increasing the driving force for chloride extrusion.

17. The method of claim 16, wherein said augmenting chloride transport within the brain comprises decreasing the extracellular concentration of potassium.

18. The method of claim 17, wherein said extracellular concentration of potassium is reduced by a ration selected from a group consisting of about 2:1, 3:1, and 5:1.

19. The method of claim 1, wherein said pathology associated with traumatic injury to the brain is prevented from occurring.

20. The method of claim 2, wherein said increasing KCC2 function comprises phosphorylating KCC2.

21. The method of claim 20, wherein KCC2 is phosphorylated at serine 940.

22. The method of claim 20, comprising the administration of a PKC activator.

23. The method of claim 22, wherein said PKC activator is phorbol 12,13-dibutyrate (PDBu).

24. A method for screening compounds for their ability to reduce the pathology associated with traumatic injury to the brain, said method comprising
a) administering a brain injury in a nonhuman animal;
b) administering a test compound to said animal either before or after said brain injury; and
c) measuring the KCC2 function within the brain, wherein an increase in KCC2 function in the animal which received the test compound as compared to the KCC2 function of an untreated animal sustaining the brain injury is indicative of the compounds ability to reduce said pathology associated with traumatic injury to the brain.

25. The method of claim 24, wherein said KCC2 function is measured in the hippocampus or dentate gyrus.

26. The method of claim 24, wherein said KCC2 function is measured by determining the protein or mRNA expression of KCC2.

27. The method of claim 24, wherein said KCC2 function is measured by determining the amount of KCC2 phosphorylation.
